(19) United States
(12) Patent Application Publication
(10) Pub. No.: US 2014/0052160 A1
(43) Pub. Date: Feb. 20, 2014

(54) OPEN SURGERY ANASTOMOSIS DEVICE, SYSTEM, AND METHOD

(71) Applicants: Devinder Singh, Baltimore, MD (US); Joshua J. White, Baltimore, MD (US)

(72) Inventors: Devinder Singh, Baltimore, MD (US); Joshua J. White, Baltimore, MD (US)

(21) Appl. No.: 13/967,275

(22) Filed: Aug. 14, 2013

Related U.S. Application Data
(60) Provisional application No. 61/684,111, filed on Aug. 16, 2012.

(51) Int. Cl. A61B 17/11

(52) U.S. Cl. CPC A61B 17/11
USPC 606/153

(57) ABSTRACT

A medical device, system and method tor an open surgical procedure configured to couple, for example, open ends of anatomical structures. The system includes an expandable tubular structure and inflatable balloon positioned within the tubular structure. The tubular structure includes a lateral opening defined in a wall of the tubular structure through which the balloon is inflated to radially expand the tubular structure, after which the balloon is deflated, and then removed from the tubular structure to, thereby, provide a fluid flow path through the tubular structure.

### CROSS-REFERENCE TO RELATED APPLICATIONS

The present application claims the benefit of U.S. Provisional Application No. 61/684,111, filed on Aug. 16,2012, the disclosure of which is hereby incorporated by reference in its entirety.

### TECHNICAL FIELD

The present invention relates generally to interconnecting tubular structures in the human anatomy in open surgical procedures and, more specifically, to anastomosis devices, systems, and methods for connecting blood vessels or other tubular structures in open surgical procedures.

### BACKGROUND

Fluid-carrying tubular structures or vessels exist in a wide number of systems, including those physiological systems found in, for example, the human body. During an open surgical procedure it is frequently necessary to modify or repair various anatomical vessels that involve the connection or anastomosis of, for example, two ends to define a fluid path. Physicians typically have employed suturing techniques to connect the ends to establish or re-establish a fluid path. However, such process is known to be time consuming and, due to the size of some vessels, such suturing often is unsatisfactory and unsuccessful. This is especially true for the anastomosis of vessels in micro-surgical procedures that require more complex and delicate suturing techniques. When dealing with small vessels, the challenge is to establish reliable fluid flow using microvascular suture techniques that provide for the anastomosis without constricting the vessels and also preventing leaks from the anastomosis or repair site. Such technically complex micro-vascular suturing techniques require a very high level of skill, and nevertheless a common occurrence is accidental capture of the vessel backwall in the suture, which then provides art unsatisfactory result in achieving a proper fluid flow path. In sum, the complex procedures and suturing techniques necessary to employ proper anastomosis between various vessels is a time consuming process and often yields unsatisfactory results. Further, the longer open surgery lasts for a patient, the higher the risk for complications to the patient.

Therefore, based on the foregoing, it would be advantageous to provide a more safe and effective means for providing an anastomosis between, for example, open ends for anatomical vessels during an open surgical procedure. Further, it would be advantageous to provide a means for anastomosis that limits or eliminates the complexity of suturing, thereby, simplifying the procedure for physicians and, therefore, limits the time required to perform anastomosis procedures to make the procedure safer for the patient.

### BRIEF SUMMARY OF THE INVENTION

The present invention is directed to systems, devices, and methods for coupling two ends of anatomical structures in an open surgical procedure. In accordance with one embodiment, a medical device for an open surgical procedure configured to couple a first end and an second end of anatomical structures is provided. The medical devices includes a tubular structure and an expandable balloon. The tubular structure includes a wall extending along a longitudinal length of the tubular structure and extending between a first open end and a second open end of the tubular structure. The wall includes a lateral opening defined in the wall and extending through the wall. The lateral opening is positioned along the wall between the first open end and the second open end of the tubular structure. The expandable balloon is configured to be positioned within the tubular structure. Further, the expandable balloon is inflatable and deflatable via fluid communication through the lateral opening of the tubular structure. In addition, the expandable balloon is removable from the lateral opening of the tubular structure.

In another embodiment, the tubular structure includes at least one of a coating and a liner member. In another embodiment, the tubular structure includes a liner member primarily positioned along an internal surface of the tubular structure with a port extension extending through the lateral opening defined in the wall of the tubular structure. In still another embodiment, the tubular structure includes an internal surface that is substantially non-thrombogenic.

In yet another embodiment, the tubular structure is configured to be radially expandable from a first position to a second position upon the expandable balloon being inflated. In another embodiment, the tubular structure includes multiple tines configured to engage the first end and second end of the anatomical structure to the tubular structure. The tubular structure, in another embodiment, includes a first portion and a second portion configured to be coupled to the first end and the second end of the anatomical structure, respectively. The multiple tines extending from the first portion of the tubular structure includes a first orientation and the multiple tines extending from the second portion of the tubular structure includes a second orientation.

In another embodiment, the expandable balloon includes a longitudinal length that extends between a first end and a second end thereof. The expandable balloon includes a balloon port for inflating and deflating the expandable balloon, in which the balloon port extends transverse to the longitudinal length of the expandable balloon and is positioned through the lateral opening of the tubular structure between the first end and the second end of the expandable balloon. In still another embodiment, the expandable balloon includes a balloon port for inflating and deflating the balloon, in which the balloon port is positioned at about a mid-point along a longitudinal length of the expandable balloon. In another embodiment, upon the expandable balloon being removed from the tubular structure, the lateral opening of the tubular structure is closed-off.

In another embodiment the tubular structure includes at least one of a metallic structure and a polymeric structure.

In accordance with another embodiment of the present invention, a medical device system for an open surgical procedure configured to couple two hollow anatomical ends is provided. The medical device system includes a tubular structure and a balloon. The tubular structure includes a wall extending along a longitudinal length of the tubular structure and extending between a first open end and a second open end of the tubular structure. The wall includes a port extension defining a lateral opening extending laterally through the wall and positioned at about a mid-point along a length of the tubular structure between the first open end and the second open end of the tubular structure. The balloon is configured to be positioned within the tubular structure. The balloon is inflatable and deflatable via fluid communication through the lateral opening defined in the wall of the tubular structure. Further, the balloon is removable from the port extension of the tubular structure.

In another embodiment, the medical device system includes a fluid flow tube that extends from the balloon and through the lateral opening defined in the port extension of the tubular structure. Further, the medical device system includes a handle with an actuator configured to control fluid flow through the fluid flow tube. In another embodiment, the medical device system includes a fluid flow source operatively coupled to the fluid flow tube, which is configured to provide fluid flow to inflate and deflate the balloon.

In another embodiment, the tubular structure radially expands upon the balloon being inflated and substantially maintains an expanded position such that the tubular structure is configured to couple to the two hollow anatomical ends upon being moved to the expanded position. In another embodiment, the port extension defining the lateral opening extends substantially transverse relative to the longitudinal length of the tabular structure. In another embodiment, the tubular structure includes a liner member primarily positioned along an internal surface of the tubular structure, the liner member defining the port extension of the tubular structure. In yet another embodiment, the port extension, upon the balloon being removed from the tubular structure, is configured to be closed-off.

In another embodiment, the tubular structure includes multiple tines sized and configured to substantially prevent the two ends of the hollow anatomical structure from migrating from the tubular structure upon the balloon being inflated to expand the tubular structure. The multiple tines, in one embodiment, include a first orientation and a second orientation relative to the wail of the tubular structure.

In another embodiment, the medical device system includes a first external structure and a second external structure each sized and configured to be positioned over separate portions of the tubular structure with me anatomical structure therebetween. Such first and second external structures may be employed to further prevent potential leaks and migration of the anatomical structure from the tubular structure.

In accordance with another embodiment of the present invention, a method for coupling a first end and a second end of hollow anatomical structures in an open surgical procedure is provided. The method includes inserting a first portion of a tubular structure into the first end of the hollow anatomical structure, the tubular structure including a wall having a longitudinal length and extending between a first open end and a second open end; inserting a second portion of the tubular structure into the second end of the hollow anatomical structure, the tubular structure including a port extension extending laterally from the wall and positioned between the first portion and the second portion of the tubular structure, the port extension defining a lateral opening extending through the wall of the tabular structure; inflating a balloon positioned within and along the longitudinal length of the tubular structure, the balloon being inflated with fluid communication through the lateral opening such that the tubular structure radially expands and engages the first and second ends of the hollow anatomical structure to the tubular structure; deflating the balloon in the tubular structure; and removing the balloon from the lateral opening of the tabular structure.

In another embodiment, the method includes closing-off the lateral opening defined in the tubular structure subsequent to removing the balloon from the tubular structure. In still another embodiment, the method step of closing-off the lateral opening in the tubular structure includes securing a clip over the port extension.

In another embodiment, the method step of inflating the balloon includes forcing fluid into the balloon through a balloon port oriented substantially transverse relative to the longitudinal length of the tabular structure and positioned within the port extension. In yet another embodiment, the method step of inflating the balloon includes radially expanding the tubular structure having multiple tines extending therefrom so that the tubular structure enlarges to a size that substantially prevents migration of the first and second ends of the hollow anatomical structure from the tubular structure to, thereby, provide a fluid flow path through the tubular structure.

In another embodiment, the method for coupling further includes securing a first external structure adjacent to the first end of the anatomical structure and over the tubular structure with the anatomical structure therebetween; and securing a second external structure adjacent to the second end of the anatomical structure and over the tubular structure with the anatomical structure therebetween. In a further embodiment, the method step of securing the first external structure includes positioning the first external structure around the anatomical structure prior to inserting the first portion of the tubular structure into the first end of the anatomical structure. Likewise, the method step of securing the second external structure includes positioning the second external structure around the anatomical structure prior to inserting the second portion of the tubular structure into the second end of the anatomical structure.

### BRIEF DESCRIPTION OF THE SEVERAL VIEWS OF THE DRAWINGS

The foregoing and other advantages of the invention will become apparent upon reading the following detailed description and upon reference to the drawings in which:

FIG. 1 is a schematic view of a medical device system including an anastomosis device with a balloon positioned therein, according to an embodiment of the present invention;

FIG. 2 is a side view of the balloon unassembled from the anastomosis device, according to the present invention;

FIG. 2A is an enlarged partial cross-sectional view taken along the longitudinal length of an upper portion of the device, depicting an extension port and tines oriented toward a mid-portion of the anastomosis device, according to another embodiment of the present invention;

FIG. 3 is a side view of the anastomosis device with the balloon adjacent open ends of tubular anatomical structures, according to an embodiment of the present invention;

FIG. 4 is a side view of two ends of the anatomical structure positioned over an anastomosis device with an fluid-flow tube extending therefrom, according to another embodiment of the present invention;

FIG. 5 is a side view of the balloon radially expanded to move the anastomosis device (shown in outline) to an expanded position within the anatomical structure, according to another embodiment of the present invention;

FIG. 6 is a side view of the balloon withdrawn from the anastomosis device and the anatomical structure, according to another embodiment of the present invention; and

FIG. 7 is a side view of two ends of the anatomical structure coupled to the anastomosis device, depicting a side access port closed-off with a clip, according to another embodiment of the present invention.

### DETAILED DESCRIPTION OF THE INVENTION

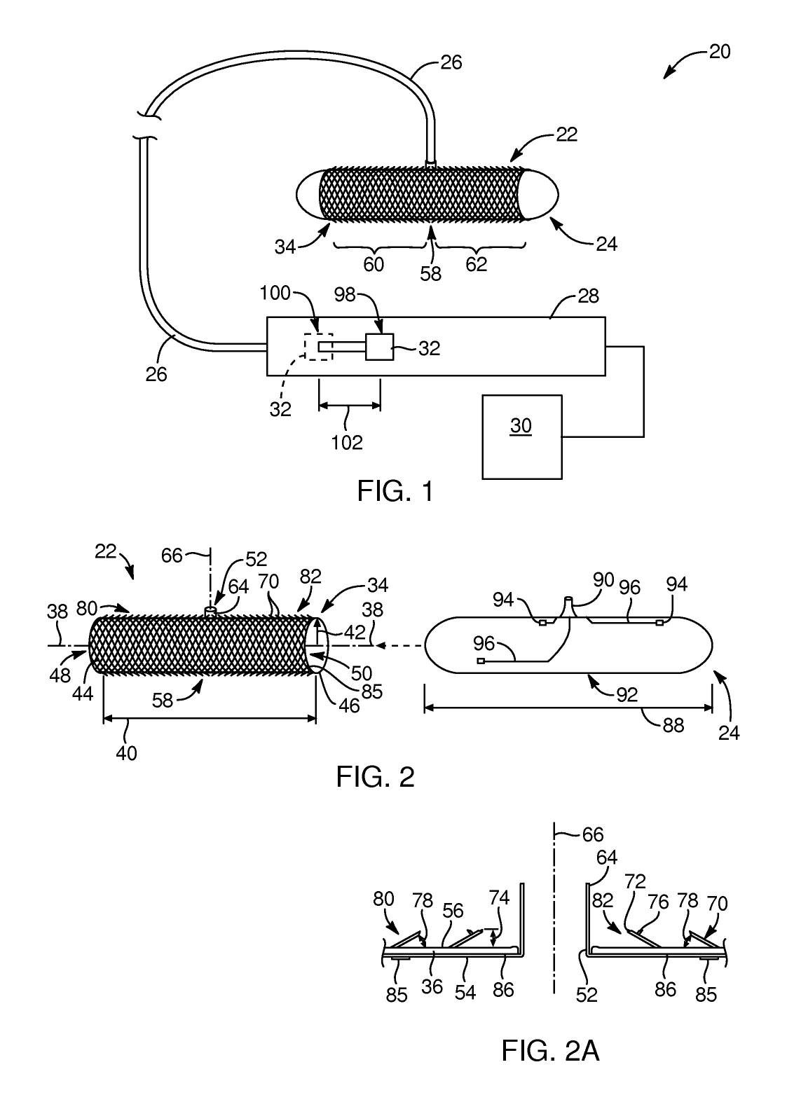

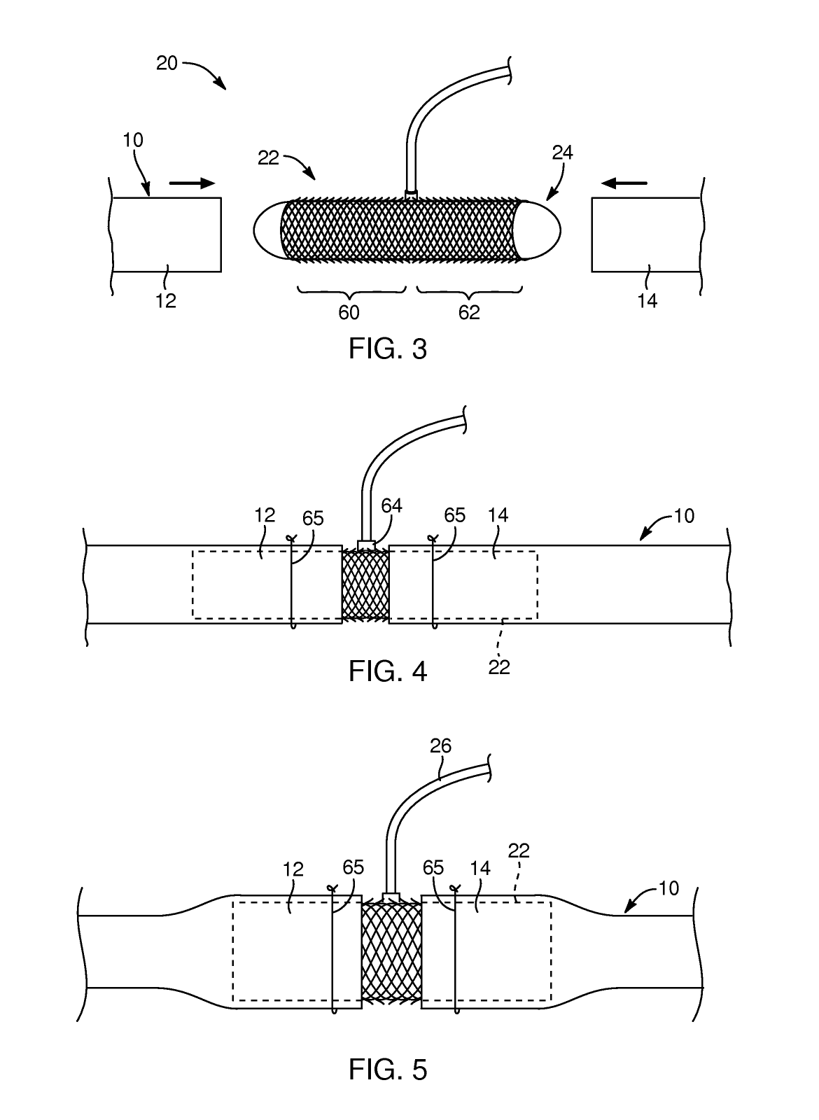

Referring to FIG. 1, a medical device system 20 configured to couple a tubular anatomical structure 10 (FIG. 3) is depicted. The medical device system 20 may include an anastomosis device 22, a balloon 24, and a fluid flow tube 26 extending between the balloon 24 and a handle 28. In addition, the handle 28 may be coupled to a fluid flow source 30. The handle 28 may include an actuator 32 configured to facilitate manual control of fluid flow between the fluid flow source and the balloon 24. The medical device system 20 of the present invention may be employed in open surgical, procedures to assist a physician in re-coupling, for example, severed (surgically divided or traumatically divided) hollow or tubular structures in the human anatomy, such as, arterial and venous vessels and/or lymphatic vessels. For example, the medical device system 20 may be employed for artery to artery coupling, vein to vein coupling, or artery to vein coupling (as well as vein to artery), or lymphatic couplings, or any other suitable tubular anatomical structures, such as that encountered in, for example, during surgical free tissue transfer, composite tissue tree flaps, and/or vascularized composite allotransplantation.

Other examples of tubular anatomical structures may include vascular structures encountered for replantation or revascularization of body parts, such as a severed digit or extremity, or solid organ transplants (allografts), such as for the kidney, pancreas, heart, lungs, liver, etc. Some other tubular structures within the urinary system may include foe ureter, bladder or urethra, or various ducts, such as in the biliary system (bile duct or its branches), or the salivary ducts, such as the parotid duct, or lymphatic ducts such as the thoracic duct. Further, other tubular structures in the human anatomy may be found in the gastrointestinal system, such as the esophagus, small bowel, large bowel, or the trachea and bronchial tree pulmonary system. Further, the reproductive system also contains tubular structures such as the male vas deferens and the female fallopian tubes, which could each require re-coupling during vasectomy reversal and/or fallopian tubal ligation reversal. Even the brain's ventricular system contains similar tabular ducts or shunts in the central nervous system. Further, the sinus system ducts, such as the frontonasal duct and the lacrimal duets are also considered tubular structures in human anatomy and could be coupled. Other tubular structures may include fetal or neonatal or pediatric or adult cardiopulmonary shunts, such as ductus arteriosus. Further, other types of tubular structures may include AV fistulas or AV grafts, and any other tubular structures encountered during peripheral vascular surgery, open endarterectomy, bypass surgery, and cardiac surgery. It should also be noted that the medical device system 20 is not limited to the human anatomy, but may be employed with other suitable mammalian anatomy for performing an anastomosis.

With reference to FIGS. 1 and 2, the anastomosis device 22 may include a tubular structure 34 or rather, a tubular shape or configuration that is defined by a wall 36. The-tubular structure includes an axis 38 extending parallel with and along a longitudinal length 40 of the tubular structure 34. Further, the tubular structure 34 includes a lateral dimension defined by a radius 42. The wall 36 of the tubular structure 34 extends between a first end 44 and a second end 46 along the longitudinal length 40 of the tubular structure 34. The first and second ends 44, 46 may each define a first open end 48 and a second open end 50, respectively. Such first and second open ends 48, 50 may include, a circular or oval configuration.

The wall 36 of the anastomosis device 22 may include an extension port 64 or side access port that defines a lateral opening 52 extending between an internal surface 54 and an external surface 56 of the tubular structure 34. The lateral opening 52 may be positioned at about a mid-portion 58 along the longitudinal length 40 of the tubular structure 34 between the first and second ends 44, 46 of the tubular structure 34 and, more particularly, between a first portion 60 and a second portion 62 of the tubular structure 34. The extension port 64 may extend substantially orthogonal or transverse from or relative to the wall 36 of the tubular structure 34. In other words, the extension port 64 that defines the lateral opening 52 or hole extending through the extension port 64 may include an opening axis 66 that is substantially orthogonal or transverse to the axis 38 of the tubular structure.

Referring to FIGS. 1, 2, and 2A, the external surface 56 of the tubular structure 34 may include multiple tines 70. In one embodiment, the tines 70 may include a point 72 sized and configured to grab and engage tissue or walls of the anatomical structure. Such tines 70 may also he sized and configured with a height 74 so as to sink into the tissue without penetrating completely through the tissue. In another embodiment, the height 74 of the tines 70 may be such so as to facilitate the tines to penetrate through the tissue. In another embodiment, the tines 70 may also include a barb 76 sized and configured to engage tissue and prevent movement of the tissue from the lines 70. In another embodiment, the tines 70 may include an atraumatic tip sized and configured to grab, but not to penetrate or sink into the tissue. Such an atraumatic tip may be bulbous or simply blunt, without a point.

The multiple tines 70 may extend in one or more directions or orientations. For example, the tines may extend substantially orthogonal to the external surface or transverse at an acute angle 78 relative to the external surface 56 of the tubular structure 34. In addition, the multiple tines 70 may be oriented with a first orientation 80 along the first portion 60 of the tubular structure 34 and may be oriented with a second orientation 82 along the second portion 62 of the tubular structure 34. In one embodiment, the tines 70 of the first orientation 80 and the second orientation 82 may each extend toward the mid-portion 58 of the tubular structure 34 at an acute angle 78. With such an orientation, the multiple tines 70 may provide ready insertion of the first and second portions 60, 62 of the tubular structure 34 Into the ends of the anatomical structure (not shown) while also providing engagement with the ends to substantially prevent migration of the ends from the tubular structure 34.

With respect to FIGS. 1 and 2, the wall 36 of the anastomosis device 22 may include a scaffold or frame like structure that may be in the form of a coil, mesh, and/or wire structure or the like that facilitates balloon expansion from a radial constricted position to a radial expanded position. For example, in one embodiment, multiple wires may be formed into a weave, a braid, or the like to form the tubular structure sized and configured to facilitate radial expansion via an inflatable balloon. In another embodiment the wall 36 or scaffold may be laser cut from a tube to form a one-piece, seamless frame having multiple interconnected struts defining multiple cells therein with, for example, diamond shaped cells that are elongated along the longitudinal length of the tubular structure. The struts defining the elongated cells may be configured to facilitate radial expansion so that the elongated cells become shorter upon inflation of a balloon. In another embodiment, the wall 36 may be laser cut from a flat sheet to form a one-piece, seamless frame having multiple interconnected struts defining elongated cells, similar to the previous embodiment. Such flat frame may then be curled and fastened together at opposite ends with, for example, fasteners, crimps or welds as known in the art, to form a tubular configuration. Further, the flat sheet material employed may be Nitinol, in which case the material may be laser cut and then heat-set to the desired tubular configuration as known by one of ordinary skill in the art.

The wall 36 may be formed from any suitable metallic and/or polymeric material, such as Nitinol, stainless steel gold, titanium, or various metallic alloys, such as, cobalt-chromium alloy, tantalum, alloy, or any other suitable material that is flexible, supportive, capable of expansion, and biocompatible as known to one of ordinary skill in the art. As set forth, the material may also include a polymeric material, such as silicone, polyethylene, polyurethane, or any other suitable polymeric materials known in the art. Further, the material may be a biodegradable or a bioabsorbable polymeric material, such as polyester, polyorthoester, and polyanhydrides, or any other suitable biodegradable or bioabsorbable polymeric material known in the art.

In another embodiment, the wall 36 or scaffolding may include markers 84. The markers 84 may be positioned on the tubular structure 34 at strategic locations to facilitate imaging of the tubular structure 34 by employing typical imaging techniques. Such imaging may be useful either during the open surgery to monitor the tubular structure 34 immediately subsequent to implantation or monitoring the position of the tubular structure 34 in follow-up visits with the physician. It also may be helpful for other physicians to readily identity the tubular structure 34 at times when the patient is being treated for an unrelated case. Materials that may be employed as markers 84 include radiopaque or radiodense materials, such as titanium, tungsten, barium, sulphate, and zirconium oxide, or any other suitable radiodense material such as a metallic/polymeric composite or the like, as known in the art.

In another embodiment, as depicted in FIGS. 2 and 2A, the anastomosis device 22 may include various device sensors 85. The device sensors 85 may be positioned at various locations along an interior and/or exterior surface of the tubular structure. The device sensors 85 may be employed to sense patency, pressure, fluid flow, oxygen, temperature, etc. to provide feedback to the physician during implantation and/or post-operative procedures. Such device sensors 85 may be wired or wireless as known by one of ordinary skill in the art.

The anastomosis device 22 may also include a coaling or liner member 86 to primarily form or cover the internal surface 54 of the anastomosis device 22 and to be employed as a shield within the tubular structure 34. Such coating or liner member 86 may also define the extension port 64 or side access port of the tubular structure 34 so as to extend through the wall 36 of the tubular structure 34 and define the lateral opening 52 and to extend laterally beyond the wall 36 of the tubular structure 34. The coating or liner member 86 may provide a substantially non-thrombogenic surface to the internal surface of the tubular structure 34 so as to be configured to substantially prevent thrombus and/or stenosis or formations that may slow or prevent flow through the anastomosis device 22. For example, the coating or liner member 86 employed to substantially prevent thrombus may be a polymeric material, such as ePTFE, or any other suitable polymeric material that will radially expand with the tubular structure 34 or stent. The liner member 86 and/or the surface of the scaffold frame may receive one or more coatings of various drugs, similar to that employed with drug-eluting stents, so as to slowly release a drug to block cell proliferation, thereby, assisting in the prevention of potential fibrosis and thrombus, or immune system modulation within the stent.

The balloon 24 may be sized and configured to be disposed or, otherwise, positioned within the tubular structure 34, or otherwise said, the anastomosis device 22. The balloon 24 may be sized and configured to be inflated to radially expand the tubular structure 34. In addition, the balloon 24 may be sized and configured to be deflated to a size so as to allow the balloon 24 to be withdrawn from the opening of the tubular structure 34. The balloon 24 may include an elongated length 88 that is equal to or longer than the length 40 of the tubular structure 34. In addition, the balloon 24 may include a side port 90 defined therein through which the balloon 24 may receive fluid flow to inflate or deflate the balloon 24. The side port 90 may be sized and configured to be positioned within and through the lateral opening 52 of the tubular structure 34. Further, the side port 90 is positioned along a mid-portion 92 of the balloon 24 to correspond with the lateral opening 52 of the tubular structure 34 such that the length 88 of the balloon 24 extends within the tubular structure 34 along its entire length 40. The side port 90 of the balloon 24 extends, and may be coupled, to the fluid flow tube 26 extending to the handle (FIG. 1). Such side port 90 may extend substantially orthogonal or transverse relative to the elongated length 88 of the balloon 24. Further, as known in the art, the balloon 24 may be inflated/deflated employing water pressure or air pressure. As such, it should be noted that the “fluid” terminology herein may be defined or refer to liquid, such as water, or air, or the like. The balloon 24 may be made from a polymeric material as known by one of ordinary skill in the art.

In another embodiment the balloon 24 may include one or more sensors 94 with conductive lines 96 extending through or along the wall of the balloon 24 and fluid flow tube 26 and provide such information to a display (now shown) coupled to, for example, the handle 28. The one or more sensors 94 may provide data and information to the physician as to the fluid pressure in the balloon 24 to minimize potential radial expansion issues on the balloon 24 as well as the tubular structure 34. Such data may also provide helpful information to the physician in determining the balloon pressure needed to properly anchor the tubular structure 34 to the anatomical structure.

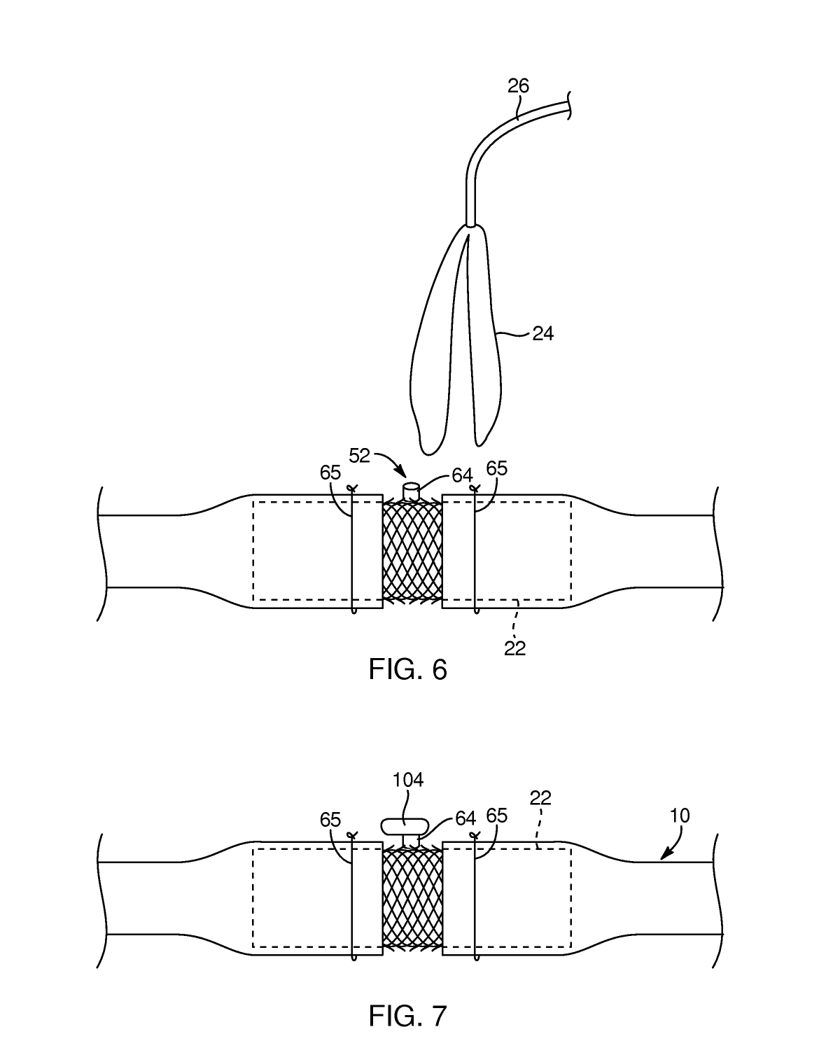

Now referring to FIGS. 3 through 7, a method for employing the medical device system 20 in an open surgical procedure is provided. With respect to FIGS. 3 and 4, the anastomosis device 22 with the balloon 24 positioned therewith is positioned adjacent a first end 12 and a second end 14 of an anatomical structure 10. The first and second ends 12, 14 of the anatomical structure 10 are tubular or hollow. The physician may manually insert the first portion 60 of the anastomosis device 22 into the first end 12 of the anatomical structure 10 and manually insert the second portion 62 of the anastomosis device 22 in the second end 14 of the anatomical structure 10. In other words, the first and second ends 12, 14 of the anatomical structure may be mounted over the first and second portions 60, 62 of the anastomosis device 22. As depicted, the first and second ends 12, 14 need only be positioned and mounted over a portion along the length of the first and second portions 60, 62 of the anastomosis device 22 so as to facilitate engagement with the tines 70 (FIGS. 2 and 2A). In another embodiment, the first and second ends 12, 14 may abut each other over the anastomosis device and/or the extension. As previously set forth, the multiple tines (not shown) of the tubular structure 34 may be oriented so m to facilitate easy insertion of the tubular structure 34 into the first and second ends 12, 14 of the anatomical structure 10. At this, stage, the physician may, if desired and as a precautionary measure, ensure securing the first and second ends 12, 14 to the anastomosis device 22 with an external structure 65 (see FIGS. 4-7). In one embodiment, the external structure 65 or external ligature may be the form of a flexible line or suture tied around the first end 12 and the second end 14 so as to assist in substantially preventing potential leaks or migration of the first and second ends 12, 14 from the anastomosis device 22. In another embodiment, the external structure 65 or external ligature may be a clamp, cuff, crimp, or ring like structure or any other suitable structure, such as a zip-tie like structure, that is biocompatible and assists in substantially preventing potential leaks or migration of the first and second ends 12, 14 from the anastomosis device 22.

In one embodiment the external structure 65 may be pre-positioned over each of the first and second ends 12, 14 of the anatomical structure 10 and, once the anastomosis device is appropriately positioned within the anatomical structure 10, the external structure 65 may be positioned and synched, crimped, tightened, or clamped over the anastomosis device 22 and the anatomical structure 10 adjacent the first and second ends 12, 14 thereof. In another embodiment. the external structure 65 may be positioned and tightened around the anatomical structure 10 adjacent the first and second ends 12, 14 thereof subsequent to positioning the anastomosis device 22 within the anatomical structure 10.

Now with reference to FIGS. 1 and 5, upon the anastomosis device 22 being positioned within the first arid second ends 12, 14 of the anatomical structure 10, the physician may now inflate the balloon 24. Such may be employed, for example, by actuating the actuator 32 on the handle 28 to allow fluid flow through the fluid flow tube 26 to inflate the balloon 24. This may be accomplished by moving the actuator 32 from the first position 100 to the second position 102 (shown in outline). Movement of the actuator 32 between the first and second positions 100, 102 facilitates fluid flow between a non-flow position and a flow position, respectively. As the balloon 24 radially expands, the anastomosis device 22 also radially expands to an enlarged, expanded position, ax shown in outline in FIG. 5. In this manner, the anastomosis device 22 may be moved between a non-expanded or constricted, first position (FIGS. 3 and 4) to a radially enlarged, expanded second position (FIG. 5). In this enlarged, position, the tines 70 (FIGS. 1 and 2A) with the first and second orientations 80, 82 may further engage the anatomical structure 10 and further prevent migration of the first and second ends 12, 14 of the anatomical structure 10 from the anastomosis device 22, as previously set forth. Furthermore, as the balloon 24 expands within the anastomosis device 22, the external structures 65 may also limit migration of the first and second ends 12, 14 from the anastomosis device 22 such that the external structures 65 become tightened (or more tightened) around the anastomosis device 22 via the balloon expanding. In one embodiment, the anastomosis device 22 may define pre-formed radially extending grooves (not shown) extending therein sized and configured to receive a respective external structure 65 with the anatomical structure 10 disposed therebetween. In this manner, the respective external structures 65 may self-seat within the radially extending grooves as the anastomosis device 22 is expanded by the balloon 24. As such, the external structures 65 may stabilize the anatomical structure 10 relative to the anastomosis device 22 to further substantially prevent migration of the device 22.

With respect to FIGS. 1, 5 and 6, once the balloon 24 is properly inflated, the actuator 32 may be moved back to the first position 98 to prevent fluid flow through the fluid flow tube 26. The fluid flow source 30 may then be changed to pull fluid flow from the balloon 24. The actuator 32 may then be moved again to the second position 100 to facilitate fluid flow through the fluid flow tube 26 to, thereby, deflate the balloon 24. Once the balloon 24 is substantially deflated, the balloon 24 may then be withdrawn or removed from the lateral opening 52 or extension port 64 of the anastomosis device 22 by manually pulling on the fluid flow tube 26 in a direction orthogonal or transverse relative to the longitudinal length 40 (FIG. 2) of the anastomosis device 22. Once the balloon 24 is withdrawn from the anastomosis device 22, the anastomosis device 22 is configured to substantially maintain its radially enlarged, expanded second position or maintain an enlarged position to facilitate continued flow through the anatomical structure 10. The physician may then manually close close-off the lateral opening 52 of the extension port 64 of the anastomosis device 22 with, for example, a fastener member 104, as depicted in FIG. 7. The fastener member 104 may be in the form of a clip or suture or any suitable structure sized and configured to be fastened or secured to the extension port 64 to close-off the lateral opening 52. With this arrangement, the medical device system 20 may he employed to implant an anastomosis device 22 to couple and maintain together, for example, severed ends of an anatomical structure 10.

While the invention may be susceptible to various modifications and alternative forms, specific embodiments have been shown by way of example in the drawings and have been described in detail herein. Further, the structural features of any one embodiment disclosed herein may be combined or replaced by any one of the structural features of another embodiment set forth herein. As such, it should be understood that the invention is not intended to be limited to the particular forms disclosed. Rather, the invention includes all modifications, equivalents, and alternatives falling within the spirit and scope of the invention as defined by the following appended claims.

What is claimed is:

1. A medical device for an open surgical procedure configured to couple a first end and a second end of anatomical structures, the medical device comprising:
a tubular structure including a wall extending along a longitudinal length of the tubular structure and extending between a first open end and a second open end, the wall including a lateral opening defined in the wall and extending laterally through the wall, the lateral opening positioned along the wall between the first open end and the second open end of the tubular structure; and
an expandable balloon configured to be positioned in the tubular structure, the expandable balloon being inflatable and deflatable via fluid communication through the lateral opening of the tubular structure, and the expandable balloon being removable from the lateral opening of the tubular structure.

2. The medical device of claim 1, wherein the tubular structure comprises at least one of a coating and a liner member.

3. The medical device of claim 1, wherein the tubular structure comprises a liner member primarily positioned along an internal surface of the tubular structure with a port extension extending through the lateral opening defined in the wall of the tubular structure.

4. The medical device of claim 1, wherein the tubular structure comprises an internal surface being substantially non-thrombogenic.

5. The medical device of claim 1, wherein the tubular structure is configured to be radially expandable from a first position to a second position upon the expandable balloon being inflated.

6. The medical device of claim 1, wherein the tubular structure comprises multiple tines configured to engage the first end and second end of the anatomical structures to the tubular structure.

7. The medical device of claim 6, wherein the tubular structure comprises a first portion and a second portion configured to be coupled to the first end and the second end of the anatomical structures, respectively, the multiple tines extending from the first portion of the tubular structure including a first orientation and the multiple tines extending from the second portion of the tubular structure including a second orientation.

8. The medical device of claim 1, wherein the expandable balloon comprises a longitudinal length extending between a first end and a second end, the expandable balloon including a balloon port for inflating and deflating the expandable balloon, the balloon port extending transverse to the longitudinal length of the expandable balloon and positioned through the lateral opening of the tubular structure between the first end and the second end of the expandable balloon.

9. The medical device of claim 1, wherein the expandable balloon comprises a balloon port for inflating and deflating the balloon, the balloon port positioned at about a mid-point along a longitudinal length of the expandable balloon.

10. The medical device of claim 1, wherein the tubular structure comprises at least one of a metallic structure and a polymeric structure.

11. The medical device of claim 1, wherein, upon the expandable balloon being removed from the tubular structure, the lateral opening of the tubular structure is closed-off.

12. A medical device system for an open surgical procedure configured to couple two hollow anatomical ends, the medical device system comprising:
a tubular structure including a wall extending along a longitudinal length of the tubular structure and extending between a first open end and a second open end, the wall including a port extension defining a lateral opening extending laterally through the wall and positioned at about a mid-point along the length of the tubular structure between the first open end and the second open end; and
a balloon configured to be positioned within the tubular structure, the balloon being inflatable and deflatable via fluid communication through the lateral opening defined in the wall of the tubular structure, and the balloon being removable from the port extension of the tubular structure.

13. The medical device system of claim 12, further comprising a fluid flow tube extending from the balloon and through the lateral opening defined in the port extension of the tubular structure.

14. The medical device system of claim 13, further comprising a handle with an actuator configured to control fluid flow through the fluid flow tube.

15. The medical device system of claim 13, further comprising a fluid flow source operatively coupled to the fluid flow tube and configured to provide fluid flow to inflate and deflate the balloon.

16. The medical device system of claim 12, wherein the tubular structure radially expands upon the balloon being inflated and substantially maintains an expanded position, the tubular structure configured to couple to the two hollow anatomical ends upon being moved to the expanded position.

17. The medical device system of claim 12, wherein the port extension defining the lateral opening extends substantially transverse relative to the longitudinal length of the tubular structure.

18. The medical device of claim 12, wherein the tubular structure comprises a liner member primarily positioned along an internal surface of the tubular structure, the liner member defining the port extension of the tubular structure.

19. The medical device system of claim 12, wherein the port extension, upon the balloon being removed from the tubular structure, is configured to be closed-off.

20. The medical device system of claim 12, wherein the tubular structure comprises multiple tines sized and configured to substantially prevent the two ends of the hollow anatomical structure from migrating from the tubular structure upon the balloon being inflated to expand the tubular structure.

21. The medical device system of claim 20, wherein the multiple tines comprise a first orientation and a second orientation relative to the wall of the tubular structure.

22. A method for coupling a first end and a second end of hollow anatomical structures in an open surgical procedure, the method comprising:
inserting a first portion of a tubular structure into the first end of the hollow anatomical structure, the tubular structure including a wall having a longitudinal length and extending between a first open end and a second open end;
inserting a second portion of the tubular structure into the second end of the hollow anatomical structure, the tubular structure including a port extension extending laterally from the wall and positioned between the first portion and the second portion of the tubular structure, the port extension defining a lateral opening extending through the wall of the tubular structure;
inflating a balloon positioned within and along the longitudinal length of the tubular structure, the balloon being inflated with fluid communication through the lateral opening such that the tubular structure radially expands and engages the first and second ends of the hollow anatomical structure to the tubular structure;
deflating the balloon in the tubular structure; and
removing the balloon from the lateral opening of the tubular structure.

23. The method according to claim 22, further comprising closing-off the lateral opening defined in the tubular structure subsequent to removing the balloon from the tubular structure.

24. The method according to claim 23, wherein the closing-off the lateral opening in the tubular structure comprises securing a clip over the port extension.

25. The method according to claim 22, wherein the inflating the balloon comprises forcing fluid into the balloon through a balloon port oriented substantially transverse relative to the longitudinal length of the tubular structure and positioned within the port extension.

26. The method according to claim 22, wherein the inflating the balloon comprises radially expanding the tubular structure having multiple tines extending therefrom so that the tubular structure enlarges to a size that substantially prevents migration of the first and second ends of the hollow anatomical structure from the tubular structure.
